SEQUENCE LISTING (6 sequences)

SEQ ID NO: 1 - protein, 7 aa, Artificial Sequence
  Synthetic Peptide C-Terminus hBD-1
GKAKCCK

SEQ ID NO: 2 - protein, 36 aa, Homo sapiens
DHYNCVSSGGQCLYSACPIFTKIQGTCYRGKAKCCK

SEQ ID NO: 3 - protein, 36 aa, Artificial Sequence
  Synthetic Peptide hBD-1 Variante 1
DHYNAVSSGGQSLYSASPIFTKIQGTSYRGKAKAAK

SEQ ID NO: 4 - protein, 36 aa, Artificial Sequence
  Synthetic Peptide hBD-1 Variante 2
DHYNSVSSGGQALYSAAPIFTKIQGTAYRGKAKSSK

SEQ ID NO: 5 - protein, 36 aa, Artificial Sequence
  Synthetic Peptide hBD-1 Variante 3
DHYNAVSSGGQALYSAAPIFTKIQGTAYRGKAKAAK

SEQ ID NO: 6 - protein, 36 aa, Artificial Sequence
  Synthetic Peptide hBD-1 Variante 4
DHYNSVSSGGQSLYSASPIFTKIQGTSYRGKAKSSK

(19) United States
(12) Patent Application Publication
(10) Pub. No.: US 2013/0336950 A1
(43) Pub. Date: Dec. 19, 2013

(54) MATERIAL COMBINATION FOR TREATING INFLAMMATORY OR INFECTIOUS DISEASES

(71) Applicants: Jan Wehkamp, Stuttgart (DE); Bjoern Schroeder, Stuttgart (DE); Eduard Stange, Stuttgart (DE)

(72) Inventors: Jan Wehkamp, Stuttgart (DE); Bjoern Schroeder, Stuttgart (DE); Eduard Stange, Stuttgart (DE)

(21) Appl. No.: 13/820,377

(22) PCT Filed: Aug. 19, 2011
(86) PCT No.: PCT/EP2011/064318

§ 371 Date: May 13, 2013

(30) Foreign Application Priority Data
Sep. 2, 2010 (DE) 10 2010 040 153.6

(51) Int. Cl. A61K 38/52; A61K 38/44; A61K 31/095; A61K 38/17

(52) U.S. Cl. CPC A61K 38/52; A61K 38/1729; A61K 38/44; A61K 38/1709; A61K 31/095
USPC 424/94.4; 514/2.3; 424/94.5; 514/2.4; 514/3.5; 424/94.1; 530/350; 514/2.8

(57) ABSTRACT

A material combination is configured to treat or prevent inflammatory and infectious diseases caused by fastidious anaerobic or facultative anaerobic microorganisms, such as bacteria or Candida sp. The material combination includes at least one antimicrobial peptide that can be activated by a reducing substance, and at least one reducing agent.

The present invention relates to a material combination for treating inflammatory and infectious human diseases that are caused by microorganisms that are obligate or facultative anaerobes, and a medicinal product containing this material combination.

Anaerobic microorganisms, especially bacteria and yeasts, i.e. unicellular microorganisms that live without oxygen or are oxygen-tolerant, can cause various clinical pictures, for example wound infections and abscesses, sepsis, infections, especially in the abdominal cavity, in the urogenital tract or in the jaw region. Thus, these pathogenic species are often already found in the region of the oral cavity, especially in the inflamed periodontium, and in the region of the stomach in the mucosal folds of the stomach, and in the duodenum, which can then cause local, but also in some circumstances systemic acute and chronic inflammations. Even in the rather thinly colonized small intestine, a number of facultative anaerobes can cause pathological changes of the highly sensitive mucosa of the small intestine; in the rectum, the principal site of the bacterial flora, admittedly aerobic bacteria predominate, but here too, anaerobic representatives are also capable of causing serious inflammatory reactions of the mucosa of the colon. Candida ssp. are also found in the stool of many individuals and are potentially pathogenic.

At present, in particular such diseases are treated with antibiotics, which mainly attack and destroy the cell walls of the bacteria. A big problem that arises when these inflammatory diseases are treated with antibiotics is the development of resistance to the antibiotics used, which in recent times has progressed even further. This enables the pathogenic bacteria/microorganisms to weaken or completely neutralize the action of antibiotic substances. If a microorganism then proves to be resistant to the common antibiotics, diseases can become life-threatening. The reason why in the past the number of multiresistant bacterial strains has increased considerably is that, owing to their rapid growth and their short culture period, the bacteria are continually able to develop new strategies for neutralizing the antibiotics. Therefore at present, in addition to antibiotics, for example also natural, especially plant, and synthetic oils and emulsions are used.

However, there is still a great need for effective possible treatments for these inflammatory diseases caused by microorganisms that are obligate or facultative anaerobes, especially bacteria or yeasts (Candida), and especially also for alternatives to antibiotics that are used for treating them.

The problem to be solved by the present invention is therefore to provide a new or alternative therapeutic approach, with which diseases caused by (an)aerobic microorganisms, incl. Candida, can be effectively treated.

This problem is solved according to the invention by a material combination that contains a) at least one antimicrobial peptide that can be activated by a reducing substance and b) at least one reducing agent, for treating or preventing inflammatory and infectious diseases that are caused by microorganisms that are obligate or facultative anaerobes, especially bacteria and/or Candida (yeasts).

The problem is further solved with a medicinal product that contains the material combination according to the invention.

With the material combination according to the invention, the aforementioned diseases and infections can be successfully combated or they can be prevented. The present inventors were able to show in this connection that the claimed antimicrobial defensins are only activated or only become antimicrobially active in combination with a reducing agent. By concomitant administration of these two ingredients, a suitable material combination is therefore provided in situ—or else through activation of the antimicrobial defensins by adding the reducing agent outside of the body and before treatment/prevention—for treating/preventing diseases that are caused by bacteria/yeasts that are obligate or facultative anaerobes.

In the present text, an “antimicrobial peptide that can be activated by a reducing substance” means any antimicrobial peptide, especially a defensin, which in its oxidized form is not antimicrobially effective, and which only acquires antimicrobial action by the action of a reducing substance. As is usual in the relevant technical area, a “peptide” means an organic compound that is derived from a chain-like linking of several amino acids into a defined sequence. It is to be understood that the peptide can be prepared naturally, i.e. isolated and purified, or can be prepared by recombinant techniques or synthetically, wherein a person skilled in the art is familiar with usual techniques and methods for preparing peptides.

A “reducing substance” or a “reducing agent”—these terms are used synonymously—means in the present text correspondingly any substance which, through provision of electrons, can break disulfide bridges in peptides and are themselves oxidized thereby. Thus, in the present text, for example the oxidoreductase thioredoxin is to be understood as a reducing substance/a reducing agent, because in its reduced form it has two SH groups, which are linked in the redox reaction with peptides to a disulfide bridge. According to the invention and according to the general understanding of a person skilled in the art in the relevant area, the reducing substance can be a chemical reducing agent, for example DTT or DTE, or also naturally occurring or recombinantly produced enzymes, such as oxidoreductases for example.

According to a preferred embodiment, a defensin is used as antimicrobial peptide that can be activated by a reducing substance.

In the present text, as is generally defined in the relevant area, “defensins” means a family of potent antimicrobial peptides, which are characterized by three disulfide bridges, and represent important key molecules of innate immunity, which protect humans against microorganisms, and which furthermore form the composition of the microbiota of mucosae. Defensins are known to be active against bacteria, fungi and viruses, and indeed it is known that they bind to their membranes and increase their permeability as a result. Based on their sequence homology and the cysteine residues present in each case, the human defensins are classified into alpha-defensins and beta-defensins.

Owing to the occurrence of defensins in human mucosae, combinatorial administration of the defensins or fragments thereof with a reducing substance, for example DTT or an oxidoreductase, is a suitable therapeutic means for all inflammatory and infectious diseases of all organs that are lined with a mucous membrane. This includes all internal organs that are in communication with the external environment, thus stomach, intestine, urinary and respiratory tracts, and the female reproductive organs. Owing to the fact that defensins occur naturally in the human mucosae, just like many oxidoreductases, as shown by the inventors, the administration of the material combination for treating/preventing inflammatory/infectious diseases of organs lined with a mucous membrane poses no risk of side effects. Owing to the ubiquitous presence of the defensins in the mucosae and the demonstrated activation thereof by reducing substances, furthermore the mechanism of action in inflammatory/infectious diseases or in prevention in the stated organs is the same.

In the present text it is to be understood that not only in each case a defensin, but also a combination of two or more defensins can be present in the material combination according to the invention.

It is further to be understood that not only the complete defensins, but also fragments thereof, which still possess the antimicrobial activity and function of the complete defensins, and can be activated by reducing substances, represent suitable antimicrobial peptides, which in the context of the present invention can be used in the material combination according to the invention. A person skilled in the art knows sufficient means and methods for finding suitable fragments of defensins, especially by C- or N-terminal shortening of the defensins, and for testing their antimicrobial efficacy. Thus, the present invention relates for example and especially also to a fragment that has between 5 and 15, preferably 7 C-terminal amino acids of defensin HBD1 (GKAKCCK; SEQ ID No. 1) or consists of these.

Correspondingly, in a preferred embodiment the at least one defensin is selected from the group comprising natural, isolated and purified defensins, recombinant defensins, synthetic defensins, or fragments thereof. For isolating naturally occurring defensins, and for producing recombinant or synthetically produced defensins, a person skilled in the art is aware of sufficient possibilities in the prior art, all of which are within his ability and knowledge, and represent usual procedures in the area in question.

In particular it is preferable if the antimicrobial defensin is selected from human beta-defensin 1 (hBD-1), human beta-defensin 2 (hBD-2), human beta-defensin 3 (hBD-3), human alpha-defensin 5 and 6, and the human neutrophil peptides 1-3. For hBD-1, which is one of the commonest peptides of its class, it was shown in connection with the present invention that in its oxidized form this had no or only slight antibiotic activity against Lactobacilli, Bifidobacteria and Candida and in contrast to this, possesses excellent antimicrobial action in its reduced form, which was brought about by simultaneous administration of thioredoxin or with chemical reducing substances.

According to another preferred embodiment, the reducing agent is an oxidoreductase, especially thioredoxin, or a chemical reducing substance, for example DTT or DTE. Suitable reducing substances are moreover thioredoxin reductase, protein disulfide-isomerase, glutaredoxin and glutathione. Thioredoxin is also a ubiquitous disulfide oxidoreductase, which through reduction of disulfide bridges of enzymes, controls and regulates their activity and therefore a number of cellular processes.

The administration of thioredoxin in inflammatory bowel diseases has admittedly already been described in U.S. Pat. No. 7,312,189, but here only the use of thioredoxin as monopreparation is proposed. The increased efficacy of a combination of thioredoxin or oxidoreductases generally and defensins is neither disclosed nor suggested in this document.

Correspondingly, the present invention also relates to the use of thioredoxin as activator of antimicrobial defensins for treating or preventing inflammatory or infectious diseases.

As already mentioned above, with the use of thioredoxin, for example in combination preparations, targeted antimicrobial activation of defensins is possible. Activation can take place by concomitant administration of thioredoxin and defensin—wherein the activation then takes place in vivo/in situ, or else by prior—ex vivo—combination, and subsequent administration of the defensin activated thereby, wherein this can optionally also be stabilized before administration.

The infectious or inflammatory diseases to be treated can be caused by a microorganism, which for example is selected from Bifidobacterium sp., Lactobacillus sp., Escherichia coli, Pseudomonas sp., and Staphylococcus sp., Streptococcus sp. or Candida sp.

Bifidobacterium sp., Lactobacillus sp., Escherichia coli, belong to the normal intestinal flora; Pseudomonas aeruginosa and Staphylococcus aureus are involved in dangerous lung diseases such as cystic fibrosis and mucoviscidosis, and Streptococcus mutans is the most important cariogenic pathogen in the oral cavity. Candida albicans can be present in the stool and is facultatively pathogenic (e.g. in immunodeficiency disorders).

It is to be understood that the above listing of particular bacterial strains is not or is not intended to be exhaustive, just like the naming of certain representatives of the individual species. To a person skilled in the art, based on the disclosure of this invention it is obvious and clear that the material combination claimed herein can be used successfully not only against these anaerobic microorganisms, but also against other anaerobic bacteria/microorganisms involved in inflammatory and infectious diseases, incl. Candida species, without it being necessary to list them here.

Correspondingly, the material combination according to the invention or thioredoxin can be used for treating or preventing diseases that are selected from chronic inflammatory bowel diseases, caries, lung diseases, diseases of the urogenital tract, diseases of the pancreas, diseases of the female reproductive tract. All these organs are characterized, as already mentioned above, by lining with mucosae, so that diseases thereof can be treated in the same way with the combination according to the invention.

It is especially preferable if the composition according to the invention is used for treating Crohn's disease or ulcerative colitis. Both diseases are chronic inflammatory bowel diseases, which can be treated with the combination of activatable defensin and oxidoreductase.

According to the invention, the material combination can be present in/as a medicinal product, and indeed in a pharmaceutically effective amount, together with a pharmaceutically acceptable carrier, diluent or excipient, and/or optionally with other pharmaceutically active substances.

Pharmaceutically acceptable carriers with optionally other additives are generally known in the prior art and are described for example in the work of Kibbe A., Handbook of Pharmaceutical Excipients, Third Edition, American Pharmaceutical Association and Pharmaceutical Press 2000. Additives comprise, according to the invention, any compound or composition that is advantageous for therapeutic use of the composition, which includes salts, binders, solvents, dispersants, and other substances usually employed in connection with the formulation of medicinal products.

The material combination/medicinal product according to the invention can be integrated in administration suitable for the respective therapy. Examples of administration comprise parenteral, e.g. intravenous, intradermal, subcutaneous, transdermal, transmucosal administrations. What amounts and what dosage forms and routes of administration are suitable in each case is within the ability and knowledge of a person skilled in the art. These can vary with respect to the patient, the specific disease and taking other factors into account.

Correspondingly, the invention also relates to a method of treating or preventing inflammatory and infectious diseases that are caused by bacteria that are obligate or facultative anaerobes, wherein the patient is administered a therapeutically effective amount of the material combination according to the invention, as described above.

It goes without saying that the aforementioned features and those yet to be further specified below are possible not only in the combination stated in each case, but also in other variations or on their own, while remaining within the scope of the present invention.

The invention is explained in more detail in the following example and in the figures, which show:

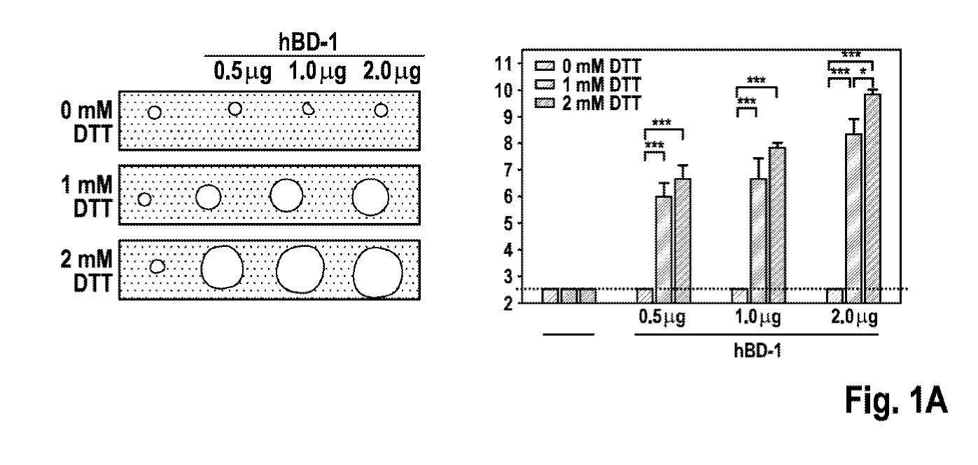

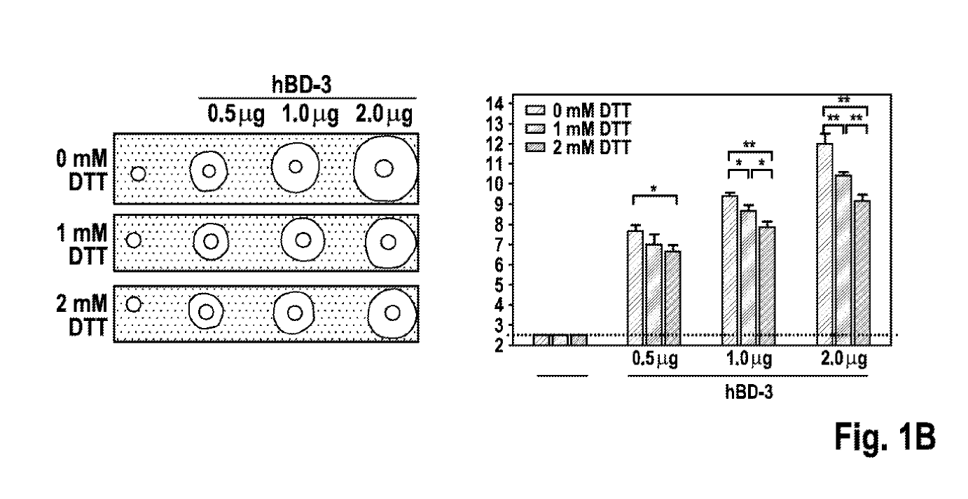

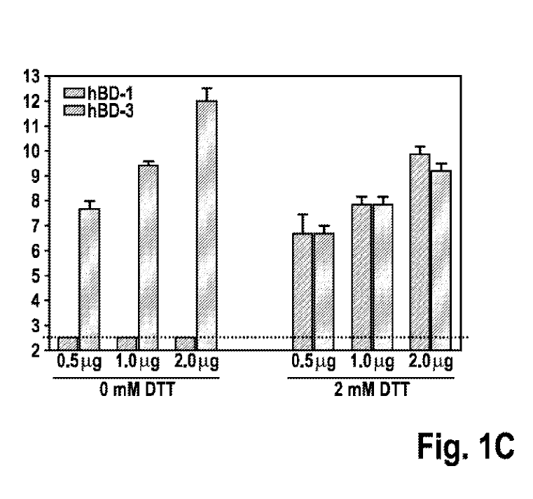

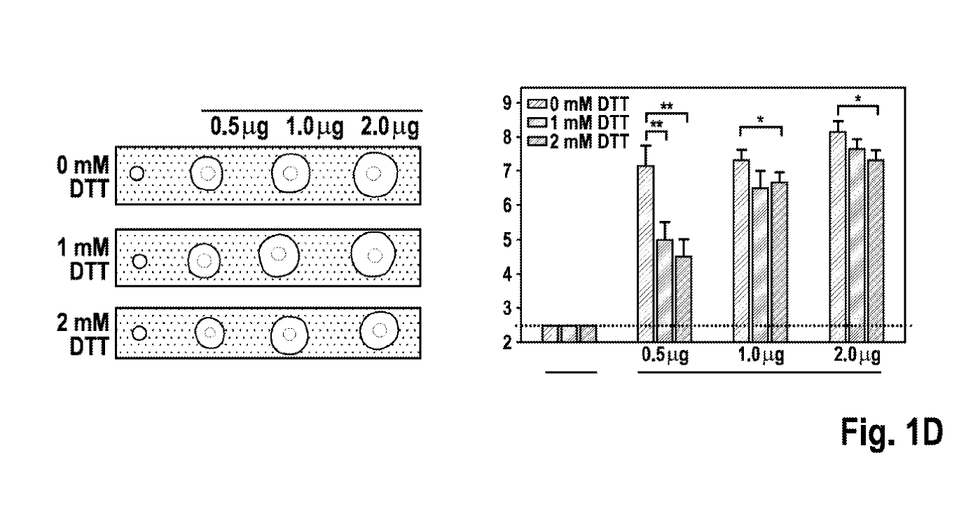

FIG. 1 the results of investigation of the antimicrobial activity of hBD-1 against Bif. adolescentis in reducing conditions; Bif. adolescentis was incubated with and without DTT (1 mM or 2 mM DTT) in anaerobic conditions with different concentrations of hBD-1 (A), hBD-3 (B) or lysozyme (D). The diameters of the inhibition zones were measured after incubation for 48 hours at 37° C. and evaluated statistically (GraphPad Prism 4.03 and Student's t-test with (*) p<0.05, (**) p<0.01, (***) p<0.001. The data are mean values, the bars represent the standard deviation; comparison of the inhibition zones between hBD-1 and hBD-3 in growth medium with or without 2 mM DTT is shown in (C); the dotted lines at 2.5 mm represent the baseline for the diameter of the wells; hBD-1 (A) shows antimicrobial activity only through reduction, hBD-3 (B) and lysozyme (D) show a slightly reduced antimicrobial activity against Bif. adolescentis in reducing conditions.

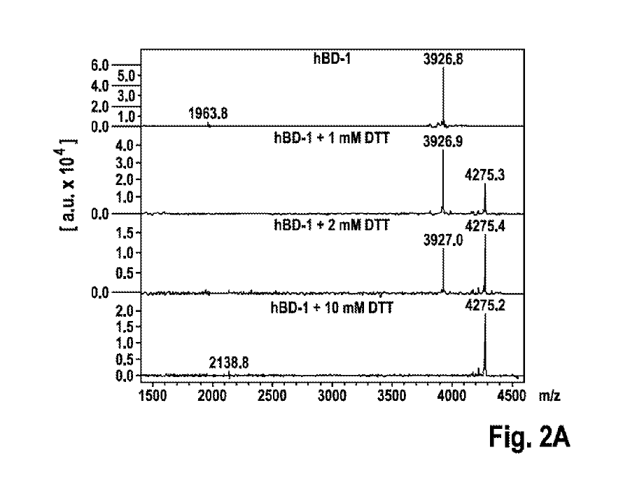

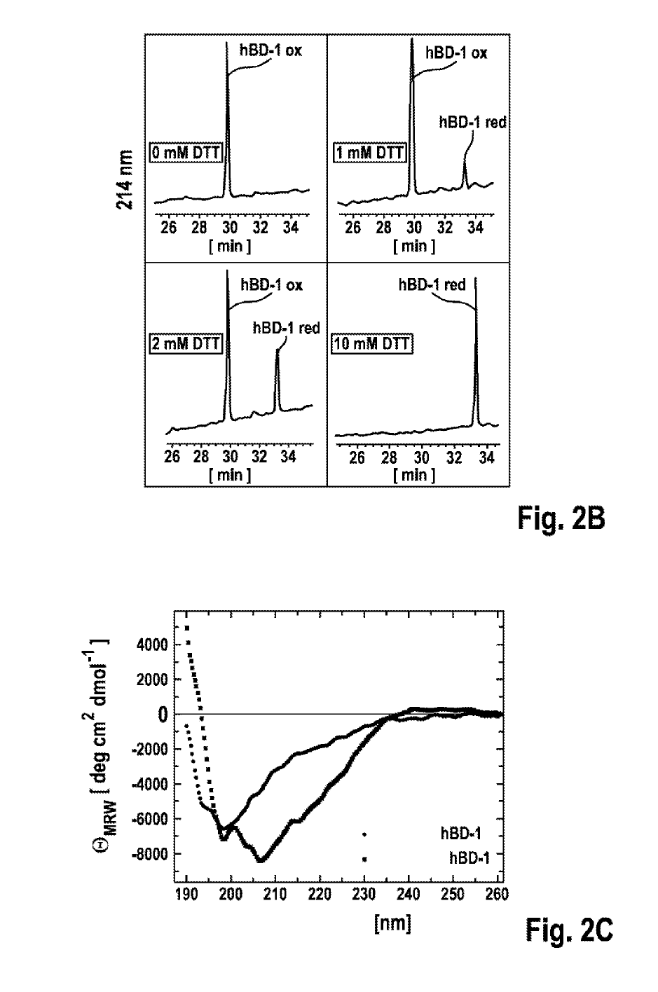

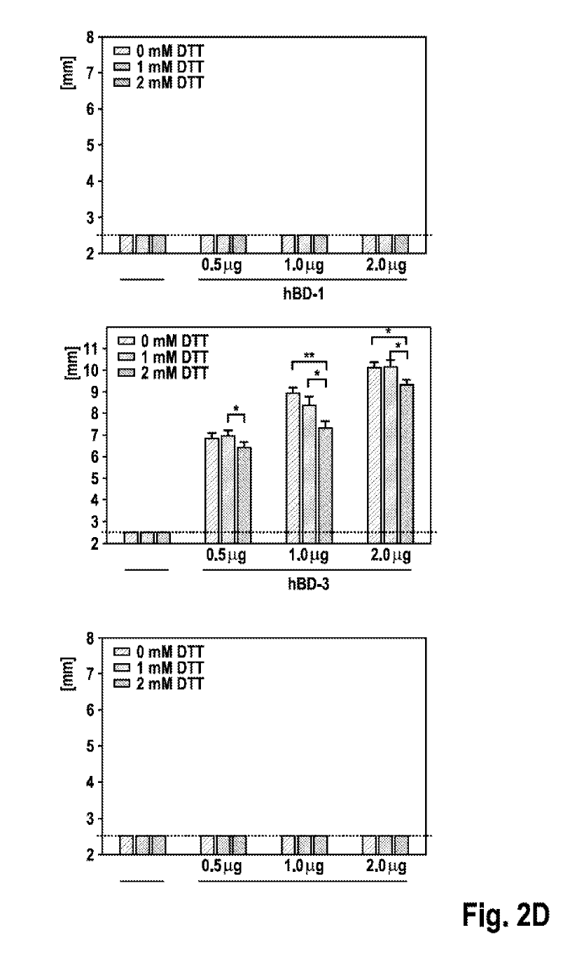

FIG. 2 the results of investigations of oxidized and of reduced hBD-1, according to which reduced hBD-1 was more hydrophobic and had a different secondary structure compared to oxidized hBD-1; (A) 1 μg hBD-1 was incubated with different concentrations of DTT, alkylated with iodoacetamide and analyzed by MALDI-MS; (B) 1 μg hBD-1 was incubated with different concentrations of DTT and analyzed by HPLC; hBD-1 ox=fully oxidized hBD-1, hBD-1 red=fully reduced hBD-1; (C) circular dichroism spectroscopy of oxidized and reduced hBD-1 (0.1 mg/ml) at 25° C. in 10 mM sodium phosphate buffer, pH 7.4; (D) in reducing conditions hBD-1 did not show any antimicrobial activity against Bacteroides vulgatus; B. vulgatus was incubated with or without DTT (1 mM or 2 mM) in anaerobic conditions with 0.5 μg, 1.0 μg or 2.0 μg hBD-1 (top diagram), hBD-3 (middle) or lysozyme (bottom diagram). After incubation for 48 h at 37° C., the diameters were measured as described for FIG. 1.

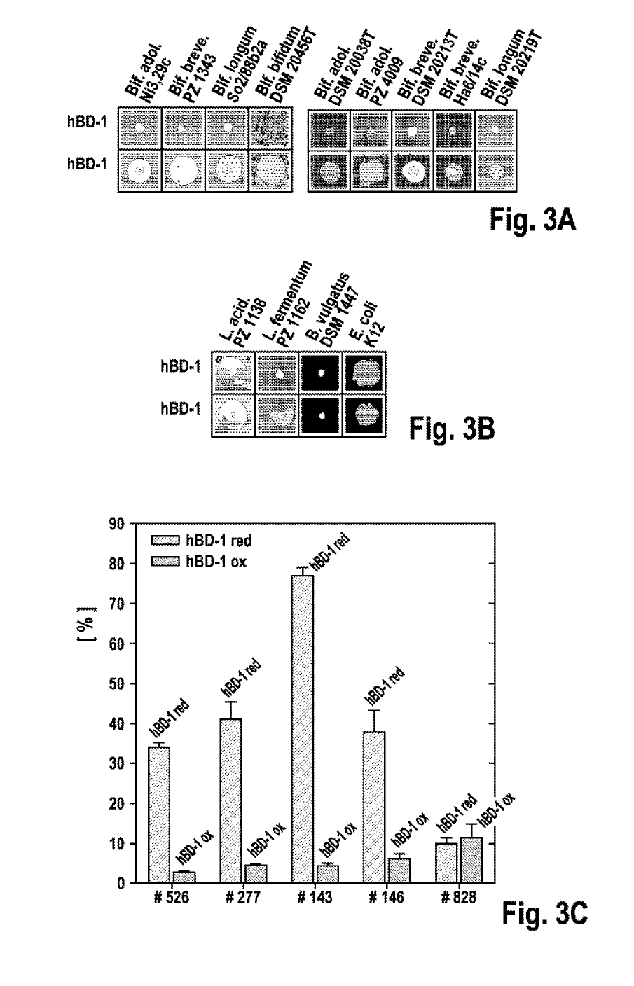

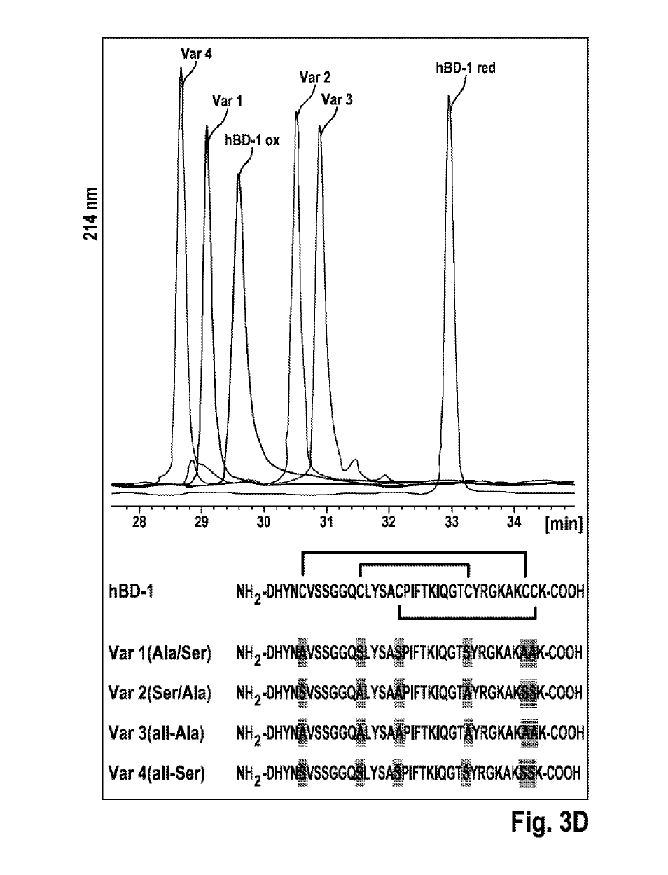

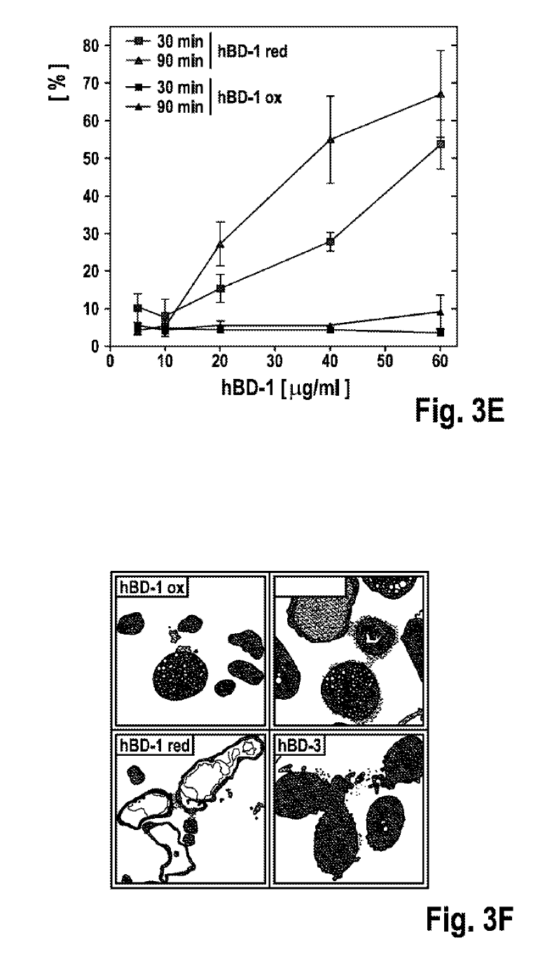

FIG. 3 Reduced hBD-1 shows bactericidal activity against various Bifidobacteria, Lactobacilli and Candida by depolarization of the cell membrane, which in Bifidobacteria ends in cellular disintegration. (A) 1.5 μg oxidized and reduced hBD-1 was incubated with Bifidobacterium spp., Lactobacillus spp., Bacteroides vulgatus (B) and Escherichia coli. For Lactobacilli, the amount of defensin was 3 μg. The formation of inhibition zones was investigated after 48 hours, representative radial diffusion assays from 3 experiments are shown. (C) reduced hBD-1 (40 μg/ml) is active against 4 out of 5 clinical Candida isolates whereas the oxidized hBD-1 shows no action. The proportion of depolarized yeasts after 90-minute incubation with reduced or oxidized hBD-1, measured with a flow cytometry assay, is shown. hBD-1 red=reduced hBD-1, hBD-1 ox=oxidized hBD-1. The mean values+standard deviation of two experiments, carried out in each case in duplicate, are shown, (D) cysteine amino acids in the hBD-1 peptide were exchanged for alanine or serine and the hydrophobicity of the hBD-1 variants was analyzed by RP-HPLC. (E) flow cytometry assay for detecting bacterial membrane depolarization of Bifidobacterium adolescentis after incubation with reduced or oxidized hBD-1. Squares show an incubation time of 30 minutes, circles a time of 90 minutes. Black symbols show oxidized hBD-1, red symbols reduced hBD-1. Results are shown as mean values±standard deviation of two experiments, in each case carried out in duplicate. (F), transmission electron microscope image of Bifidobacterium adolescentis after incubation for 2 hours with 200 μg/ml oxidized hBD-1 (hBD-1 ox, top left), without defensin (control, top right), with 200 μg/ml reduced hBD-1 (hBD-1 red, bottom left) or with 200 μg/ml hBD-3 (bottom right). Fixed bacteria were analyzed by transmission microscopy, magnification bars=200 nm.

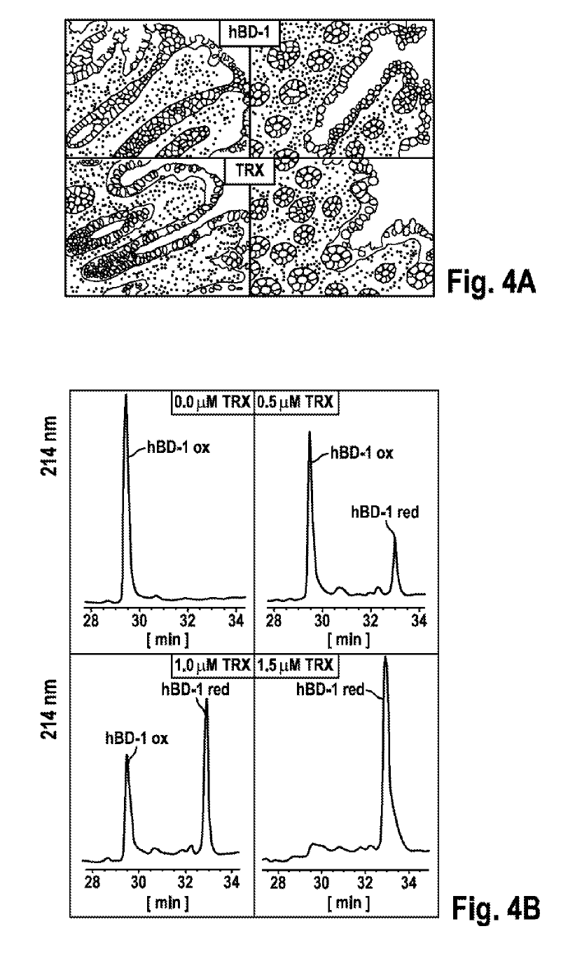

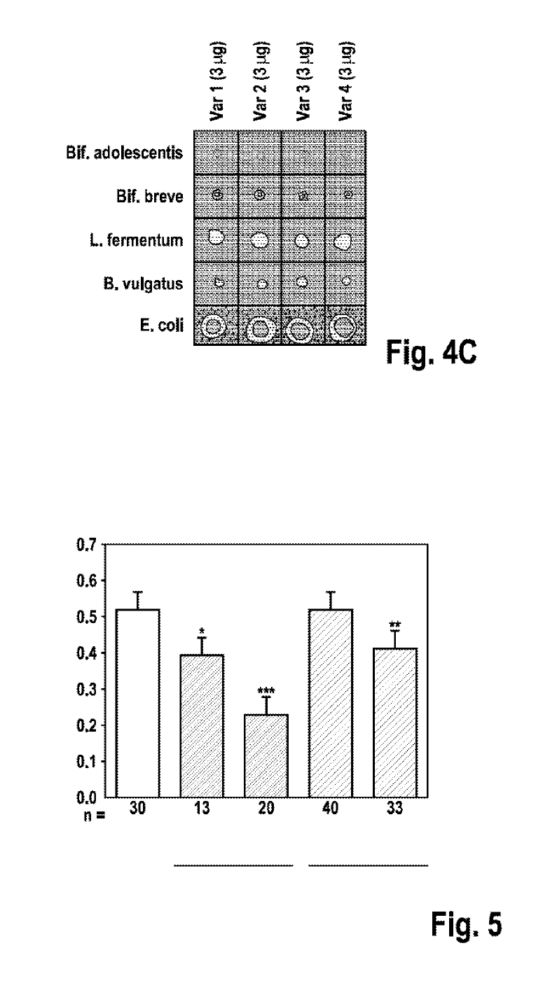

FIG. 4 Thioredoxin (TRX) co-localized with hBD-1 and is capable of catalyzing the reduction of hBD-1. (A) immuno-histochemistry staining of hBD-1 and thioredoxin of human colon sections. Magnification bars=100 μm. (B) 10 μM oxidized hBD-1 was incubated with or without 0.5, 1.0 and 1.5 μM human thioredoxin and 100 nM rat thioredoxin reductase for 30 minutes at 37° C. in 0.10 M potassium phosphate-2 mM EDTA, pH 7.0 buffer and 0.8 mM NADPH. Incubation assays were analyzed by RP-HPLC and the transformation of oxidized to reduced hBD-1 was observed. hBD-1 ox=oxidized hBD-1, hBD-1 red=reduced hBD-1. (D) Antimicrobial activity of the Ala/Ser variants of hBD-1 (3 μg) against Bifidobacterium adolescentis Ni3,29c, Bif. breve PZ 1343, Lactobacillus fermentum PZ 1162, Bacteroides vulgatus DSM1447 and Escherichia coli K12, measured with the radial diffusion assay. Formation of inhibition zones was investigated after incubation for 48 hours, representative assays are shown.

FIG. 5 Expression of thioredoxin mRNA in human colon biopsies of healthy control patients and patients with chronic inflammatory bowel diseases (uninflamed and inflamed). Thioredoxin mRNA expression was analyzed by real-time PCR, compared with healthy controls and normalized to beta-actin. The data were evaluated statistically with GraphPad Prism 4.03 and the Mann-Whitney test with (*) p<0.05, (**) p<0.01 and (***) p<0.001. Data are shown as mean values±standard deviation.

### EXAMPLES

Reduction of various peptides leads to their antimicrobial activity.

Materials and Methods:

The bacterial strains used were obtained from Ardeypharm (Herdecke, Germany) and from the German Collection for Microorganisms and Cell Cultures (DSMZ, Brunswick, Germany).

The assay (antimicrobial radial diffusion assay) with respect to antimicrobial activity was carried out as follows: The bacteria were cultured anaerobically before the antimicrobial test. During execution of the assay in 10 mM sodium phosphate with 0.3 mg/ml of trypticase soya broth (TSB) and 1% (w/v) agarose with low EEO (Sigma-Aldrich), incubation took place in anaerobic conditions with antimicrobial peptides and 0-2 mM DTT. The presence of a reducing environment was controlled by adding the redox indicator resazurin. After 48-h incubation, the diameter of the inhibition zones was measured and was evaluated statistically using GraphPad Prism 4.03 and Student's t-test.

Matrix-supported laser desorption/ionization mass spectroscopy (MALDI-MS) was carried out as follows: hBD-1 was incubated with DTT, alkylated with iodoacetamide and co-crystallized with alpha-cyano-4-hydroxycinnamic acid. MALDI-MS was carried out with a Bruker Ultraflex TOF/TOF machine.

For the high-performance liquid chromatography (HPLC) analysis, a system of the Agilent 1200 series and a Vydac 218TP-C18 column were used, using a water/acetonitrile gradient with trifluoroacetic acid as ion-pair reagent.

Circular dichroism (CD) spectroscopy was carried out on a Jasco J-715 spectropolarimeter (Jasco Corporation, Japan) at 25° C. in 10 mM sodium phosphate, pH 7.4. The spectra were recorded at 0.1 mg/ml in the far-UV region.

The thioredoxin reductase assay was carried out as follows: For this, oxidized hBD-1 was incubated with 0.5-1.5 μM thioredoxin or protein disulfide-isomerase in the presence of 100 nM thioredoxin reductase and 0.8 mM NADPH in 0.1 M calcium phosphate buffer-2 mM EDTA. The incubation mixtures were analyzed by RP-HPLC, with which the transformation of oxidized to reduced hBD-1 peptide could be observed.

Flow-cytometry antimicrobial assay, in which the membrane depolarization of the bacteria was measured, was carried out as described in: Nuding, S., et al., “Antibacterial activity of human defensins on anaerobic intestinal bacterial species: a major role of HBD-3.” Microbes. Infect. 11 (3), 384-393 (2009).

For transmission electron microscopy of the bacteria, approx. 2×10⁸ CFU (colony forming units) of Bifidobacterium adolescentis Ni3,29c were incubated anaerobically for two hours at 37° C. in the absence or in the presence of 200 μg/ml synthetic defensin. The bacteria were centrifuged, fixed in prewarmed Karnovsky's solution and stored at 4° C. for 24 hours. After centrifugation, the pellet was embedded in 3.5% agarose at 37° C., coagulated at room temperature, cut into small blocks and fixed in Karnovsky's solution again. The post-fixing was based on 1.0% osmium tetroxide with 1.5% potassium ferrocyanide in 0.1 M cacodylate buffer. After embedding in glycidyl ether, the blocks were cut with an ultramicrotome (Ultracut, Reichert, Vienna, Austria). Ultrathin sections (30 nm) were applied on copper grids and analyzed with a Zeiss LIBRA 120 transmission electron microscope (Carl Zeiss, Oberkochen, Germany) at 120 kV.

Immunohistochemical staining was carried out with sections of human intestine biopsies embedded in paraffin. Rabbit-anti-hBD1 was diluted 1:500; rabbit anti-TRX was diluted 1:1000. The anti-rabbit secondary antibody was peroxidase-coupled, and detection was carried out with a DAKO detection system.

Isolation of total RNA and real-time PCR: The measurement of thioredoxin mRNA in human intestine biopsies is described in: Wehkamp, J., et al., “NOD2 (CARD15) mutations in Crohn's disease are associated with diminished mucosal alpha-defensin expression.” Gut 53 (11), 1658-1664 (2004).

Results:

The classical radial diffusion assay (as described in Lehrer, R. I., et al., “Ultrasensitive assays for endogenous antimicrobial polypeptides.” J. Immunol. Methods 137 (2), 167-173 (1991)) was modified, in order to analyze the antimicrobial activity of synthetic human beta-defensin 1 (hBD-1) against the bacterial strains of the anaerobic normal flora, including the Gram-positive strain Bifidobacterium adolescentis and the Gram-negative strain Bacteroides vulgatus, in anaerobic conditions. Small concentrations of the reducing agent dithiothreitol (DTT) were added to the assay medium, whereas medium without DTT served as control. After incubation for 48 hours, the antimicrobial activity was determined by measuring the diameters of the inhibition zones. In the medium without DTT, different amounts of hBD-1 had no effect on the growth of Bifidobacterium adolescentis (FIG. 1A).

In the medium with 1 mM DTT, concentration-dependent inhibition zones were detected. This effect was dose-dependent, as increasing DTT concentrations led to an increase of the inhibition zones in size and definition. In order to rule out an effect of DTT on bacterial fitness, the same tests were performed with beta-defensin hBD-3 and lysozyme. hBD-3 showed strong antimicrobial activity in the medium without DTT and reduced activity after adding DTT (FIG. 1B); a similar effect was observed for lysozyme (FIG. 1D). This shows that the increased antimicrobial activity of hBD-1 in a reducing environment was a specific effect, which could not be attributed to impaired bacterial fitness. At concentrations of 2 mM DTT, hBD-1 was as effective as hBD-3 against Bif. adolescentis (FIG. 10). This result is surprising, as hBD-3 is one of the strongest antimicrobial peptides in oxygen-rich environments, whereas hBD-1 is classified as one of the weakest. In a test with the Gram-negative anaerobic strain Bacteroides vulgatus (see FIG. 2D), a growth-inhibiting effect was not observed in any of the conditions, whereas hBD-3 showed concentration-dependent inhibition zones.

As beta-defensins are characterized by three intramolecular disulfide bridges, the involvement of the cysteine bridges in the antimicrobial action was investigated next. For this, hBD-1 was incubated with increasing concentrations of DTT, and the samples were analyzed by MALDI. A signal was observed for the oxidized hBD-1 (calculated monoisotopic mass: 3926.8 Da, FIG. 2A). After incubation with increasing concentrations of DTT and carboxamide methylation, in order to detect free sulfhydryl (SH) groups, a second peak with a mass difference of +348 dalton was detected, which corresponded to the fully reduced form with six alkylated cysteine residues (—CH₂CONH₂, +58 dalton). These results suggest an all-or-nothing mechanism, in which all three disulfide bridges are either present or not, without the possibility of an intermediate stage with one or two disulfide bridges. Consequently, after incubation with DTT a fraction of hBD-1 was reduced completely, whereas the second fraction remained oxidized.

Next, a reversed-phase high performance liquid chromatography (RP-HPLC) analysis was carried out, to verify the quantitative results of MALDI-MS (FIG. 2B). Whereas oxidized hBD-1 possessing all disulfide bridges eluted after 30 minutes, incubation of the hBD-1 peptide with increasing concentrations of DTT resulted in a shift toward a second peak at 33.5 minutes, which corresponded to the fully reduced hBD-1 peptide (which was confirmed by mass spectroscopy, data not shown). In RP-HPLC, therefore the reduced hBD-1 showed an increase in retention time, which pointed to an increase in the hydrophobicity of the linear peptide in solution.

Next, the secondary structure was investigated using circular dichroism (CD) spectroscopy (FIG. 2C). The native hBD-1 peptide showed a characteristic minimum at 209 nm for alpha-helices and a significant signal at 218 nm, which corresponded to the content of beta-pleated sheet, and which indicated a well-folded peptide. In contrast to this, the reduced peptide did not show any of these minima, but a minimum at about 195 nm, which indicated a change of conformation to a random-coil structure. In fact, calculation of the secondary structure elements with various algorithms for the reduced peptide suggests an increase in the random-coil fraction. Therefore the antimicrobial activity of hBD-1 in a reducing environment was attributed to its reduced, open form.

In order to rule out that the observed effects can be attributed to changes of the bacterial surface, caused by the reducing agent DTT in the medium, the antimicrobial activity of the oxidized and of the reduced hBD-1 against Bifidobacterium adolescentis, Bif. breve, Bif. longum and Bif. bifidum was tested in anaerobic conditions in medium without DTT. The oxidized form of hBD-1 did not show any antimicrobial activity against strains of Bifidobacterium spp. (FIG. 3A). In contrast, the reduced hBD-1 peptide showed antimicrobial activity against all the strains tested (FIG. 3A), whereas the size of the inhibition zones varied among the strains. Furthermore, the same assay was carried out with the Gram-positive strains Lactobacillus acidophilus and L. fermentum, and with the Gram-negative strain Bacteroides vulgatus and the facultatively anaerobic strain Escherichia coli K12 (FIG. 3B). Reduced hBD-1 led to clear inhibition zones with the Lactobacilli, whereas oxidized hBD-1 showed no effect at all. For the Gram-negative strain Bacteroides vulgatus, no effect could be observed for any of the hBD-1, but for E. coli, antimicrobial activity of both the oxidized and the reduced form was observed. This showed that hBD-1 is specifically activated by a reducing environment against Gram-positive anaerobes of the human normal flora. In the test with respect to the facultatively pathogenic fungus Candida albicans, an effective antifungal activity against four out of five strains could be detected for the reduced hBD-1, but not for the oxidized hBD-1 (FIG. 3C). This shows that hBD-1 is activated by a reducing environment not only against Gram-positive anaerobes of the human normal flora, but also against a medically-important facultatively pathogenic fungus.

Next, Ala/Ser variants of hBD-1 were generated, in which all cysteine residues were exchanged with alanine or serine residues, in order to investigate whether a permanently open structure is responsible for the increased activity (FIG. 3D). However, these variants were either inactive or only very weakly active against Bifidobacteria and Lactobacilli, using amounts of peptide of up to 3 μg (FIG. 4C).

The only bacterium that was sensitive to variants 1 to 4 was E. coli K12, which was also sensitive to the oxidized hBD-1 (FIG. 3B). For investigating the hydrophobicity of the generated Ala/Ser variants, RP-HPLC analyses were carried out (FIG. 3D). All four variants were—compared to reduced hBD-1—eluted at lower acetonitrile concentrations, which indicated lower surface hydrophobicity. A shortened variant of hBD-1, which did not have the seven C-terminal amino acids GKAKCCK (hBD-1_{Δ30-36}) (SEQ ID No. 1), was unable to inhibit the growth of Bif. adolescentis in normal conditions or in reducing conditions (data not shown).

Furthermore, the antimicrobial activity of the complete hBD-1 disappeared in the presence of 150 mM sodium chloride (data not shown). Therefore a linear structure alone is not sufficient for the antimicrobial activity of reduced hBD-1, rather it is the hydrophobicity, ion interactions and free cysteine residues in the C-terminus that are important.

In order to investigate whether the observed inhibition of growth of Gram-positive anaerobes is bacteriostatic or bactericidal, flow-cytometry antimicrobial assays were carried out with bacterial membrane depolarization as the reading system. This confirmed the results of the radial diffusion assay, according to which the reduced hBD-1 peptide was more effective against Bif. adolescentis, compared with the oxidized form (FIG. 3E). The reduced peptide caused membrane depolarization in 50% of the bacteria after only 30 minutes of incubation with 60 μg/μl, whereas the oxidized peptide showed no significant depolarization at this time point or after 90 minutes. This pointed to a rapid bactericidal effect of the linear hBD-1.

Next, transmission electron microscopy was carried out with Bifidobacterium adolescentis, to analyze the activity of the linear hBD-1 on a morphological basis. After incubation with oxidized hBD-1 (FIG. 3F, top left), the bacterial cells did not show morphological damage of any kind, compared with the untreated control (top right). In contrast, after incubation with reduced hBD-1 (bottom left), cellular disintegration could be observed, whereas the membrane structures remained morphologically intact. This process started intracellularly, before it spread to the whole bacterium, and led to complete loss of the cytoplasmic contents. This once again indicated that reduced hBD-1 had bactericidal rather than bacteriostatic action, and in fact by a mechanism different from hBD-3, which mainly attacks the bacterial membrane (bottom right).

Physiological, enzymatic redox regulation is mainly controlled by thioredoxin (TRX), a multifunctional oxidoreductase. As TRX is expressed by mucosae and has extracellular functions, TRX expression as well as hBD-1 expression in human intestinal tissue was investigated by means of immunohistochemical tests. As shown in FIG. 4A, hBD-1 could be detected in the intestinal mucosa, including the epithelium lining the intestinal crypts (top). A similar staining pattern was obtained for thioredoxin (FIG. 4A, top), indicating that both hBD-1 and thioredoxin are co-localized in vivo.

In order to analyze whether hBD-1 is a natural substrate for thioredoxin, hBD-1 was incubated with different concentrations of TRX in the presence of NADPH and thioredoxin reductase, which together represent the natural thioredoxin system. After incubation, the samples were analyzed by RP-HPLC, to investigate the oxidized and reduced hBD-1 fractions. As shown in FIG. 4b, hBD-1 was reduced by thioredoxin, the process being concentration-dependent. Increasing TRX reductase also increased the amount of reduced hBD-1, whereas omission of either TRX or TRX reductase prevented reduction of hBD-1 (data not shown). The catalyzing transformation of oxidized hBD-1 to reduced hBD-1 can therefore be attributed to the thioredoxin system. Low oxygen levels of less than 1% can occur in wounds and infectious tissue. These sites are very amenable to attack, for invasion by anaerobic pathogens and by bacteria that colonize the surrounding epithelium.

Bifidobacteria are Gram-positive anaerobic microorganisms, which inhabit the human gut as a major component of the normal flora. Lactobacilli are also found in the human gut, in the vagina, and in the oral/pharyngeal cavity, all of which represent localizations with a low oxygen content.

The present data show that a change in environmental conditions has a considerable influence on the antimicrobial activity of hBD-1.

The present data further show that hBD-1 in an anaerobic, reducing environment protects the mucosal epithelium against colonization by the bacteria of the normal flora. Through activation in the lumen, which depends on the environmental conditions, effective antimicrobial activity can be triggered.

1. A material combination, comprising:
at least one antimicrobial peptide that is activatable by a reducing agent; and
at least one reducing agent, wherein the material combination is therapeutically effective in treating or preventing inflammatory and infectious diseases caused by obligate or facultative anaerobes.

2. The material combination as claimed in claim 1, wherein the at least one antimicrobial peptide is a defensin or fragments thereof.

3. The material combination as claimed in claim 2, wherein the defensin or the fragments thereof are selected from a group including one of:
natural, isolated and purified defensins,
recombinant defensins,
synthetic defensins,
fragments of natural, isolated and purified defensins,
fragments of recombinant defensins, and
fragments or synthetic defensins.

4. The material combination as claimed in claim 2, wherein the defensin is selected from human beta-defensin 1, human alpha-defensin 6, a human neutrophil peptide, fragments of human beta-defensin 1, fragments of human alpha-defensin 6, and fragments of a human neutrophil peptide.

5. The material combination as claimed in claim 1, wherein the at least one reducing agent is selected from one of chemical reducing agents and enzymatic reducing agents.

6. The material combination as claimed in claim 1, wherein the at least one reducing agent is an oxidoreductase.

7. The material combination as claimed in claim 1, wherein the at least one reducing agent is selected from thioredoxin, thioredoxin reductase, DTT, DTE, protein disulfide-isomerase, glutaredoxin and glutathione.

8. A thioredoxin operable to activate an antimicrobial peptide, wherein the activation results in a therapeutically effective reagent to treat or prevent inflammatory or infectious diseases.

9. The thioredoxin as claimed in claim 8, wherein the thioredoxin activates the antimicrobial peptides via one of in vivo activation and in vitro activation.

10. A material combination, comprising:
at least one antimicrobial peptide that is activatable by a reducing agent; and
at least one reducing agent, wherein:
the material combination is therapeutically effective in reducing an inflammatory response to an inflammatory or infectious disease that is caused by obligate or facultative anaerobes,
thioredoxin is used to activate the at least one antimicrobial peptide to reduce the inflammatory response, and
the inflammatory or infectious disease is caused by a microorganism, which is a bacterium or a yeast.

11. The material combination as claimed in claim 10, wherein the inflammatory or infectious disease is selected from Bifidobacterium sp., Lactobacillus sp., Escherichia coli, and Candida sp.

12. The material combination as claimed in claim 10, wherein the inflammatory or infectious disease is selected from chronic inflammatory bowel diseases, caries, lung diseases, diseases of the urogenital tract, diseases of the pancreas, and diseases of the female reproductive tract.

13. The material combination as claimed in claim 12, wherein the chronic inflammatory bowel disease is selected from Crohn's disease and ulcerative colitis.

14. A medicinal product, comprising:
a material combination including:
at least one antimicrobial peptide activatable by a reducing agent, and
at least one reducing agent, wherein the material combination is therapeutically effective to reduce the inflammatory response caused by obligate or facultative anaerobe infection in a mammal; and
a pharmaceutically acceptable carrier.
